(19) United States
(12) Patent Application Publication
(10) Pub. No.: US 2024/0374185 A1
(43) Pub. Date: Nov. 14, 2024

(54) HEMOLYSIS-REDUCTION CONNECTOR FOR DIRECT BLOOD DRAW

(71) Applicant: CAREFUSION 303, INC., San Diego, CA (US)

(72) Inventors: Erik RUNDLETT, Brea, CA (US); Abin AUSTIN, Thrissur (IN); Manjunath MANGUMALI, Bangalore (IN); Sumit Raosaheb MANE, Ahmednagar (IN); Sudharshan MUNDADISHETTY, Udupi Dist (IN)

(21) Appl. No.: 18/315,155

(22) Filed: May 10, 2023

(51) Int. Cl. A61B 5/15; A61M 39/10

(52) U.S. Cl. CPC A61B 5/150946; A61M 39/10

(57) ABSTRACT

A flow restriction device may include a male luer connector portion and a female luer connector portion. The male luer connector portion can define a first lumen and the female luer connector portion can define a second lumen. A flexible tubing preferably provides fluid communication between the first lumen and second lumen. The flexible tubing can be helically wound around a solid core, and the flexible tubing can be helically wound within a hollow chamber. The flexible tubing is configured to increase flow resistance through the tubing to reduce shear stress on the fluid, thereby decreasing the incidence of hemolysis.

### BACKGROUND

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter is an over-the-needle peripheral intravenous (“IV”) catheter (PIVC). As its name implies, the over-the-needle catheter may be mounted over an introducer needle having a sharp distal tip. A catheter assembly may include a catheter hub, the catheter extending distally from the catheter hub, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

For blood withdrawal or collecting a blood sample from a patient, a blood collection container may be used. The blood collection container may include a syringe. Alternatively, the blood collection container may include a test tube with a rubber stopper at one end. In some instances, the test tube has had all or a portion of air removed from the test tube so pressure within the test tube is lower than ambient pressure. Such a blood collection container is often referred to as an internal vacuum or a vacuum tube. A commonly used blood collection container is a VACUTAINER® blood collection tube, available from Becton Dickinson & Company.

The blood collection container may be coupled to the catheter. When the blood collection container is coupled to the catheter, a pressure in the vein is higher than a pressure in the blood collection container, which pushes blood into the blood collection container, thus filling the blood collection container with blood. A vacuum within the blood collection container decreases as the blood collection container fills, until the pressure in the blood collection container equalizes with the pressure in the vein, and the flow of blood stops.

Unfortunately, as blood is drawn into the blood collection container, red blood cells may experience high shear stress and be susceptible to hemolysis due to the flow rate through and geometry of the blood collection system. Hemolysis may result in rejection and discard of a blood sample. The blood draw can also result in an area of local negative pressure at the catheter tip, which may lead to catheter tip collapse, vein collapse, or other complications that prevent or restrict blood from filling the blood collection container. Furthermore, blood spillage during and/or after blood draw may occur.

The description provided in the background section should not be assumed to be prior art merely because it is mentioned in or associated with the background section. The background section may include information that describes one or more aspects of the subject technology.

### SUMMARY

Provided herein are embodiments of flow restriction devices that are configured to reduce the incidence of hemolysis. In some embodiments, the flow restriction devices comprise a male luer connector portion defining a first lumen; a female luer connector portion defining a second lumen; and a middle connector portion comprising a flexible tubing with a first end and a second end, the first end configured to fluidly couple with the first lumen and the second end configured to fluidly couple with the second lumen; wherein the tubing is helically arranged between the male luer connector portion and the female luer connector portion and comprises a fluid passage that is configured to provide fluid communication between the first lumen and the second lumen.

Some embodiments provide a flow restriction devices that comprise a first connector portion defining a first lumen; a second connector portion defining a second lumen; and a third connector portion comprising a tubing with a first end and a second end, the first end configured to fluidly couple with the first lumen and the second end configured to fluidly couple with the second lumen, wherein the tubing comprises a fluid passage that is configured to provide fluid communication between the first lumen and the second lumen.

In some embodiments, peripheral intravenous catheter assemblies are provided that are configured to limit hemolysis during drawing of blood from a patient. Some embodiments of these assemblies comprise a catheter hub having a proximal end and a distal end; a fluid collection device; and a flow restriction device, comprising: a male luer connector portion defining a first lumen; a female luer connector portion defining a second lumen; and a middle connector portion comprising a flexible tubing with a first end and a second end, the first end configured to fluidly couple with the first lumen and the second end configured to fluidly couple with the second lumen; wherein the tubing is arranged between the male luer connector portion and the female luer connector portion and comprises a fluid passage that (i) is configured to provide fluid communication between the first lumen and the second lumen and (ii) comprises a length and a diameter that is configured to increase flow resistance through the tubing to reduce shear stress on the fluid.

The present disclosure provides a flow restriction device, comprising: a male luer connector portion defining a cavity and a first lumen in fluid-flow continuity with the cavity; and a female luer connector portion defining a second lumen and an extension in fluid-flow continuity with the second lumen, the extension comprising: a first end; a second end; an outer surface; and a channel defined on the outer surface, wherein the channel is helically disposed around the outer surface of the extension and extends between the first and second ends, the extension is configured to be at least partially disposed within the cavity, and the channel and the inner surface of the cavity define a fluid passage in fluid-flow continuity with the first lumen and the second lumen.

In some instances, the present disclosure provides a flow restriction device, comprising: a male luer connector portion defining a first cavity and a first lumen in fluid-flow continuity with the first cavity; and a female luer connector portion defining a second lumen and an extension, wherein the extension defines a second cavity in fluid-flow continuity with the second lumen, the extension comprising: a first end; a second end; an outer surface; and a channel defined on the outer surface, wherein the channel is helically disposed around the outer surface of the extension and extends between the first and second ends, the extension is configured to be at least partially disposed within the first cavity, and the channel and the inner surface of the first cavity define a fluid passage in fluid-flow continuity with the first cavity and the second cavity.

In some aspects, the present disclosure provides a peripheral intravenous catheter assembly configured to limit hemolysis during drawing of blood from a patient, comprising: a catheter hub having a proximal end and a distal end; a fluid collection device; and a flow restriction device, comprising: a male luer connector portion defining a cavity and a first lumen in fluid-flow continuity with the cavity and the catheter hub; and a female luer connector portion defining a second lumen in fluid-flow continuity with the fluid collection device and an extension in fluid-flow continuity with the second lumen, the extension comprising: a first end; a second end; an outer surface; and a channel defined on the outer surface, wherein the channel is helically disposed around the outer surface of the extension and extends between the first and second ends, the extension is configured to be at least partially disposed within the cavity, and the channel and the inner surface of the cavity define a fluid passage in fluid-flow continuity with the first lumen and the second lumen.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The following figures are included to illustrate certain aspects of the embodiments and should not be viewed as exclusive embodiments. The subject matter disclosed is capable of considerable modifications, alterations, combinations, and equivalents in form and function, as will occur to those skilled in the art and having the benefit of this disclosure.

FIG. 1 illustrates a vascular access device including a peripheral intravenous catheter (PIVC) assembly that includes a flow restriction device, in accordance with some embodiments of the present disclosure.

FIG. 2 illustrates a perspective view of the flow restriction device, in accordance with some embodiments of the present disclosure.

FIG. 3 illustrates an exploded view of the flow restriction device of FIG. 2, in accordance with some embodiments of the present disclosure.

FIG. 4 illustrates an exploded cross-sectional view of the flow restriction device of FIG. 2, in accordance with some embodiments of the present disclosure.

FIG. 5A illustrates a perspective view of a flow restriction device, in accordance with some embodiments of the present disclosure.

FIG. 5B illustrates an exploded partial cross-sectional view of the flow restriction device of FIG. 5A, in accordance with some embodiments of the present disclosure.

FIG. 6A illustrates a perspective view of a flow restriction device, in accordance with some embodiments of the present disclosure.

FIG. 6B illustrates an exploded view of the flow restriction device of FIG. 6A, in accordance with some embodiments of the present disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

Blood draw via a vascular access device has drawn increasing attention attributed to minimized needle sticks and improved operation efficiency as compared with traditional blood draw methods with venipuncture. Current blood draw using a peripheral intravenous catheter (PIVC) has seen some challenges, one of the most critical is hemolysis related blood quality. In particular, with currently existing PIVC products in the market, along with the standard connection (such as a short extension set and a needleless connector), and blood collection devices (such as a Vacutainer), the shear stress exerted onto red blood cells often causes hemolysis.

Various embodiments of the present disclosure are directed to providing systems and methods to address hemolysis in PIVC blood draw with a hemolysis reduction accessory (also referred to herein as a flow restriction device), such as a connector, which is attached to the PIVC and serves as a flow restrictor to reduce risk of hemolysis. The hemolysis-reduction accessory is advantageously compatible with PIVC placement and does not necessitate change to any of the existing operations. The hemolysis-reduction accessory of the various embodiments described herein is potentially applicable to a wide variety of PIVC products, and compatible with existing blood collection devices and infusion disposables.

Various embodiments of the present disclosure focus on effective flow restriction with the add-on hemolysis-reduction accessory (also referred to herein as a flow restriction device) that regulates the overall flow rate of the entire fluid path as blood travels through. The flow restriction device can be either assembled with the PIVC or co-packaged with the PIVC. The clinician may connect a blood collection device to the port of the accessory and can then draw blood to the intended volume. After blood draw, the clinician may disconnect and discard the flow restriction device and the blood collection device together. As such, this flow restriction device can be either for single blood draw or stay inline throughout indwell.

The flow restriction devices and associated blood collection systems of the various embodiments described herein additionally provide further advantages over currently existing blood collection systems. For example, add-on flow restriction devices described herein reduce hemolysis while allowing blood to be drawn through an already-placed PIVC. Further, the flow restriction devices described herein are compatible with PIVC connections and allow for seamless blood draw at insertion. Additionally, since the flow restriction devices are an add-on which can be easily incorporated without any changes to existing PIVC, there is minimal impact to clinical setting and operations.

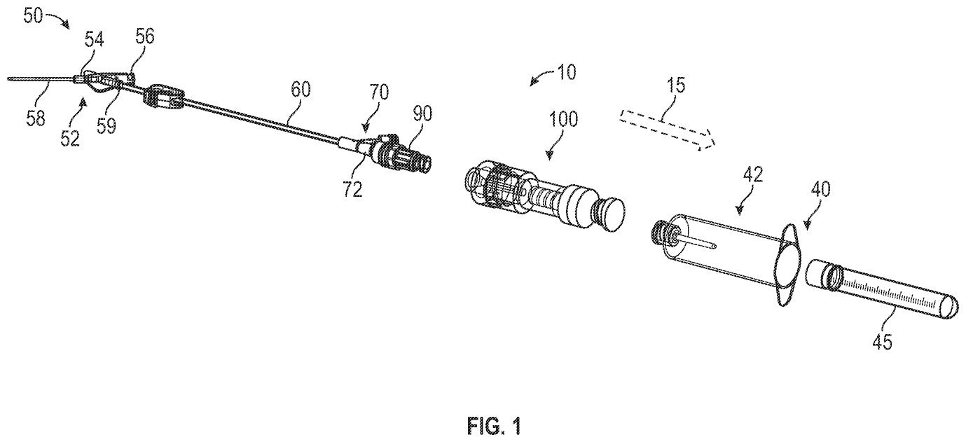

FIG. 1 illustrates a vascular access device 10 including a peripheral intravenous catheter (PIVC) assembly 50 that includes a connector or flow restriction device 100, in accordance with some embodiments of the present disclosure. The flow restriction device 100 may be configured to facilitate connections for components of the vascular access device 10 and reduce a likelihood of hemolysis during blood collection using the vascular access device 10. In some embodiments, the vascular access device 10 may include a catheter assembly 50. The catheter assembly 50 may include a catheter hub 52, which may include a distal end 54, a proximal end 56, and a lumen extending through the distal end and the proximal end. The catheter assembly 50 may further include a catheter 58, which may be secured within the catheter hub 52 and may extend distally from the distal end 54 of the catheter hub 52. In some embodiments, the catheter assembly 50 may be a peripheral intravenous catheter (PIVC).

In some embodiments, the catheter assembly 50 may include or correspond to any suitable catheter assembly 50. In some embodiments, the catheter assembly 50 may be integrated and include an extension tube 60, which may extend from and be integrated with a side port 59 of the catheter hub 52. A non-limiting example of an integrated catheter assembly is the BD NEXIVA™ Closed IV Catheter system, available from Becton Dickinson and Company. In some embodiments, a proximal end of the extension tube 60 may be coupled to an adapter, such as, for example, a Y-adapter 70. In some embodiments, the flow restriction device 100 may be fluidly coupled to the Y-adapter 70.

In some embodiments, the catheter assembly 50 may be non-integrated and may not include the extension tube 60. In these and other embodiments, the flow restriction device 100 may be configured to couple to the proximal end 56 of the catheter hub 52 or another suitable portion of the catheter assembly 50. In some embodiments, the flow restriction device 100 may be coupled directly to the catheter assembly 50, eliminating the extension tube 60 and providing a compact catheter system.

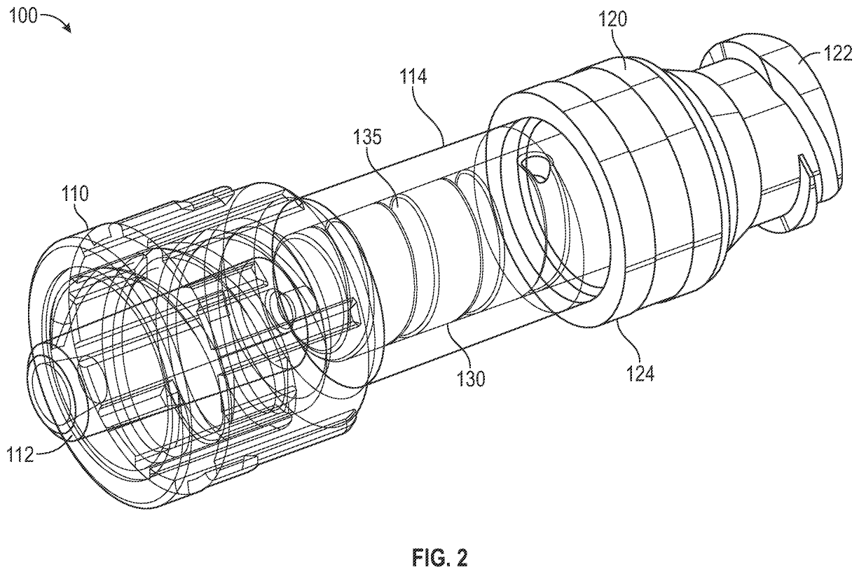

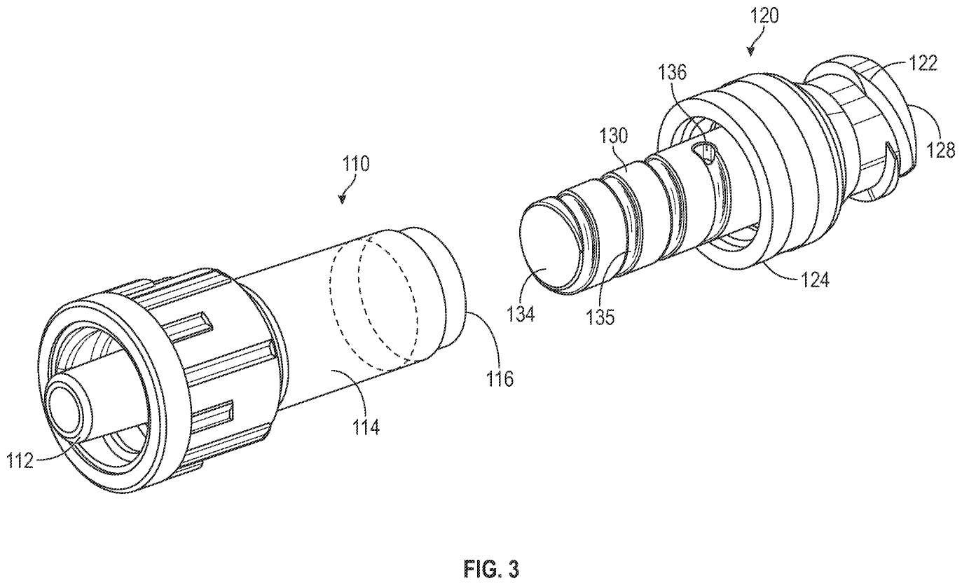

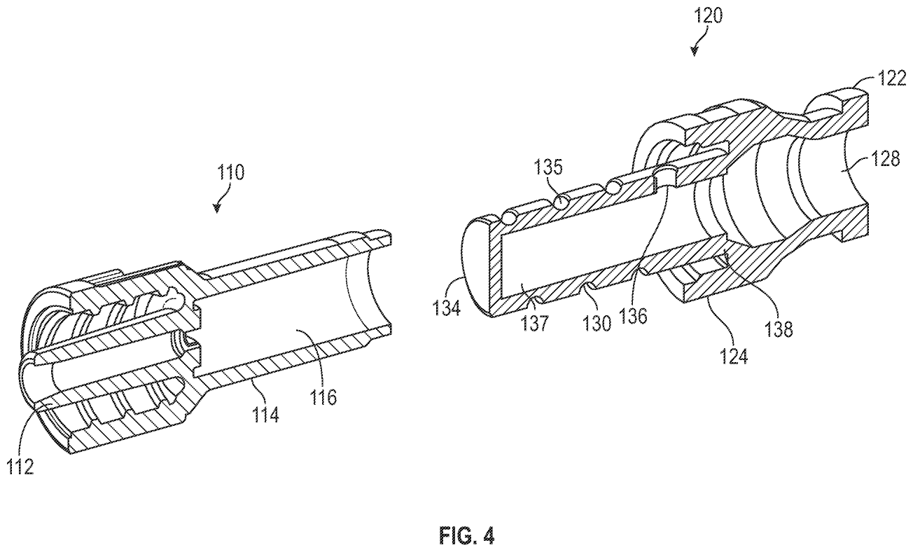

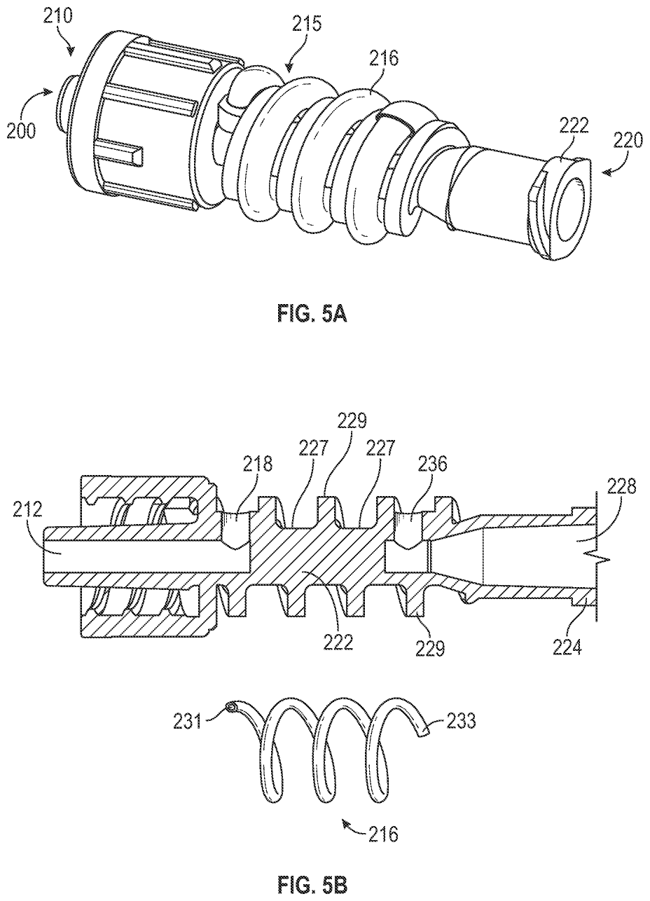

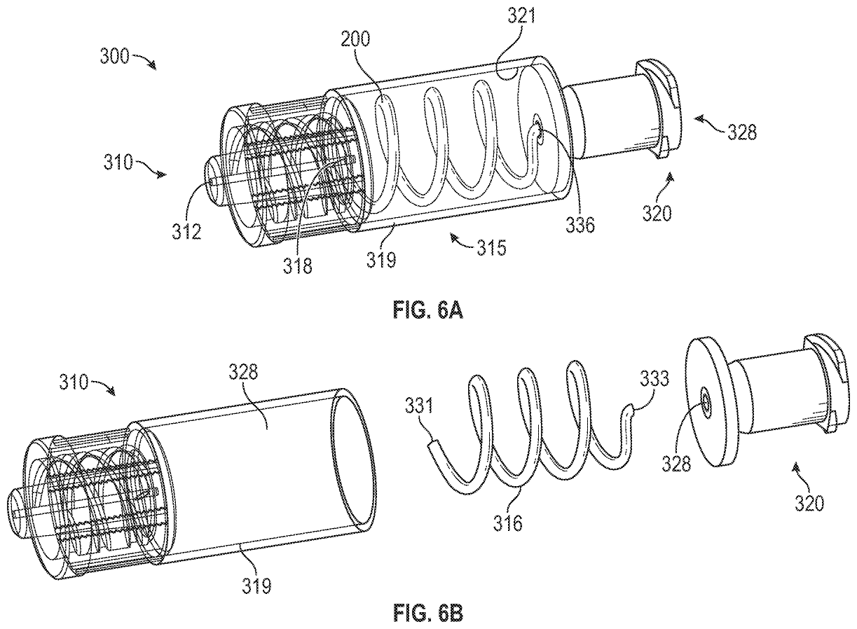

FIG. 2 illustrates a perspective view of the flow restriction device 100, in accordance with some embodiments of the present disclosure. FIG. 3 illustrates an exploded view of the flow restriction device 100 of FIG. 2, in accordance with some embodiments of the present disclosure. FIG. 4 illustrates an exploded cross-sectional view of the flow restriction device 100 of FIG. 2, in accordance with some embodiments of the present disclosure. FIG. 5A illustrates a perspective view of a flow restriction device, in accordance with some embodiments of the present disclosure. FIG. 5B illustrates an exploded partial cross-sectional view of the flow restriction device of FIG. 5A, in accordance with some embodiments of the present disclosure. FIG. 6A illustrates a perspective view of a flow restriction device, in accordance with some embodiments of the present disclosure. FIG. 6B illustrates an exploded view of the flow restriction device of FIG. 6A, in accordance with some embodiments of the present disclosure.

As illustrated in FIGS. 2-4, and with continued reference to FIG. 1, in some embodiments, the flow restriction device 100 may include a male luer connector portion 110 configured to couple to the catheter assembly 50. The male luer connector portion 110 may have an internal surface defining a lumen 112 of the male luer connector portion 110. In some embodiments, a distal end of the male luer connector portion 110 can be coupled to the catheter assembly 50. In some embodiments, the lumen 112 is in fluid-flow continuity with a cavity 116 defined by the body 114 of the male luer connector portion 110.

In some embodiments, the flow restriction device 100 may further include a female luer connector portion 120 disposed proximally of the male luer connector portion 110. The female luer connector portion 120 may be configured to couple to fluid collection device 40 (e.g., a blood collection device). For example, the female luer connector portion 120 may be integrated with the blood collection device 40 or monolithically formed with the blood collection device 40 as a single unit or piece. As another example, the female luer connector portion 120 may be in the form of a female luer connector or another suitable connector, which may be coupled with a male luer portion of the blood collection device 40. The female luer connector portion 120 may include an internal surface defining a lumen 128 extending therethrough for coupling to the male luer portion of the blood collection device 40. In some embodiments, a proximal end of the female luer connector portion 120 can be coupled to the blood collection device 40. Optionally, a threaded portion 122 of the female luer connector portion 120 can be utilized to engage the female luer connector portion 120 to a corresponding male luer portion.

In the depicted example, the body 114 of the male luer connector portion 110 can be coupled to the body 124 of the female luer connector portion 120 to allow fluid-flow continuity between the lumen 112 and the lumen 128 of the flow restriction device 100 and form a common housing.

In the depicted example, the female luer connector portion 120 can include an extension 130 to control fluid flow between the lumens 112, 128 and reduce hemolysis through the total blood flow pathway from PIVC to blood collection device 40. As illustrated, the extension 130 can be disposed within the cavity 116 formed by the male luer connector portion 110. As illustrated, the extension 130 directs fluid flow between the lumens 112 and 128 of the male luer connector portion 110 and the female luer connector portion 120, respectively while inducing a resistance to the fluid flow. By inducing a resistance to the fluid flow, the rate of fluid flow through the assembly from PIVC to blood collection device is reduced. Reducing the flow rate of a fluid, such as blood, through the assembly can reduce the hemolysis index of the blood.

In some embodiments, an extension 130 defines a groove or channel 135 that directs fluid flow from a distal end 134 of the extension 130 to a proximal end 138 of the extension 130. As illustrated, the channel 135 can be defined along an outer surface of the extension 130. Accordingly, when the extension 130 is positioned within the cavity 116 of the male luer connector portion 110, at least a portion of the outer surface of the extension 130 can engage against an inner surface of the cavity 116 to permit the channel 135 and the inner surface of the cavity 116 to cooperatively define a fluid passage therebetween. The defined fluid passage can extend along a path between the distal end 134 and the proximal end 138 of the extension 130 to reduce a rate of fluid flow across the extension 130. In some embodiments, the groove or channel 135 can be formed on an inner surface of the body 114 such that engagement with the outer surface of the extension 130 forms a fluid pathway. In some embodiments, the groove or channel 135 can extend along one or both of the inner surface of the body 114 and the outer surface of the extension 130 such that when the body 114 receives the extension 130, a fluid pathway is formed to conduct fluid between the body 114 and the extension 130.

In the depicted example, fluid flow can pass from the lumen 112 of the male luer connector portion 110 to the distal end 134 of the extension 130 via the cavity 116. In some embodiments, fluid can flow from the proximal end 138 of the extension 130 to the lumen 128 of the female luer connector portion 120 via a port 136 formed through the extension 130.

In some embodiments, the port 136 can allow fluid flow between the proximal end 138 of the extension 130 and/or the channel 135 into a cavity 137 formed within the extension 130. The cavity 137 formed within the extension 130 can be separated from the cavity 116 of the male luer connector portion 110 by the body of the extension 130. As illustrated, the cavity 137 can be in fluid-flow continuity with the lumen 128. Therefore, in some embodiments, fluid flow can pass from the proximal end 138 of the extension 130 to the lumen 128 via a port 136 and the cavity 137.

Therefore, in some embodiments, the channel 135 (in conjunction with the inner surface of the cavity 116) can direct fluid flow from the male luer connector portion 110 into the fluid collection device 40 via the female luer connector portion 120. As depicted, the channel 135 may provide a fluid pathway from the catheter assembly 50 to the fluid collection device 40 via the flow restriction device 100. For example, in some embodiments, a leg 72 of the Y-adapter 70 may be coupled to the flow restriction device 100. The leg 72 of Y-adapter 70 may include a lumen into which the distal end of the male luer connector portion 110 may be coupled. The Y-adapter 70 may provide fluid-flow continuity between the flow restriction device 100 via a connector 90, which is depicted as a needleless connector, and the channel 135 with the catheter assembly 50, for example, via the extension tubing 60. Accordingly, the channel 135 may define a fluid pathway with a helical, spiral, extended, or otherwise indirect path (as discussed below) through which fluid entering the flow restriction device 100 from the catheter assembly 50, may flow through the flow restriction device 100 for collection in the fluid collection device 40. For example, where blood is being withdrawn or collected from a patient, the medical fluid may be blood, and the fluid collection device 40 may be a blood collection device. In some embodiments, the blood collection device may be a blood collection tube holder such as the BD Vacutainer® LuerLok™ Access Device (LLAD). Accordingly, during blood collection or withdrawal from the patients, the blood sample may flow from the distal end of the male luer connector portion 110 into the LLAD 40 via the flowpath or channel 135.

In some embodiments, the extension 130 can define a longitudinal axis that extends through the distal and proximal ends 134, 138. The channel 135 spans between the distal and proximal ends 134, 138 of the extension 130, and is configured to spiral or helically extend along the longitudinal axis. Because the path spirals about the longitudinal axis, the channel 135 extends indirectly between the distal and proximal ends 134, 138 of the extension 130. The indirect path between the distal and proximal ends 134, 138 of the extension 130 can have a length that is greater than a distance between the distal and proximal ends 134, 138 of the extension 130. In some instances, the pitch and the angle of the turns of the helical channel 135 can be optimized to reduce the volumetric flow rate of a fluid, e.g., blood, moving through the channel 135 by inducing resistance to the fluid. Since the decreased blood flow rate results in a reduction in shear stress experienced by the red blood cells, the risk of hemolysis during blood collection may advantageously be reduced.

As illustrated in FIGS. 5A-5B, and with continued reference to FIGS. 1-4, in some embodiments, a flow restriction device 200 may include a male luer connector portion 210 configured to couple to the catheter assembly 50. The flow restriction device also includes a middle connector portion 215 and a female luer connector portion 220. The male luer connector portion 210 may have an internal surface defining a lumen 212 of the male luer connector portion 210. In some embodiments, a distal end of the male luer connector portion 210 can be coupled to the catheter assembly 50. In some embodiments, the lumen 212 is in fluid-flow continuity, or fluid communication, with a tubing 216 through a first port 218.

The tubing 216 is preferably a flexible tubing that is capable of being manipulated and wound around a core 222 of the middle connector portion 215. The winding of the tubing 216 around the middle connector portion 215 can be guided or secured by exterior contours of the core 222. For example, the middle connector portion 215 can include a helical groove that, in cross-section, creates a series of troughs 227 and ridges 229. The tubing 216 can be wound about the core 222 nestled in the groove or troughs 227, with the ridges 229 extending between or along adjacent coils of the tubing 216.

In some embodiments, the female luer connector portion 220 is disposed proximally of the male luer connector portion 210. The female luer connector portion 220 may be configured to couple to fluid collection device 40 (e.g., a blood collection device). For example, the female luer connector portion 220 may be integrated with the blood collection device 40 or monolithically formed with the blood collection device 40 as a single unit or piece. As another example, the female luer connector portion 220 may be in the form of a female luer connector or another suitable connector, which may be coupled with a male luer portion of the blood collection device 40. The female luer connector portion 220 may include an internal surface defining a lumen 228 extending through the female luer connector portion 220 for fluid communication between the male and female luer connector portions 210, 220. In some embodiments, the female luer connector portion 220 may include a threaded portion 224 that can be utilized to couple the female luer connector portion 220 to a corresponding male luer portion.

In the depicted example, the tubing 216 can fluidly couple the lumen 212 of the male luer connector portion 210 with the lumen 228 of the female luer connector portion 220. FIG. 5B illustrates an exploded view of the flow restriction device 200. As illustrated, the tubing 216 includes a first end 231 and a second end 233. The first end 231 is preferably configured to be coupled to the first port 218 of the male luer connector portion 210. The first end 231 may be sized to fit within the first port 218 and secured by adhesive, ultrasonic welding, or some other means of coupling. In some embodiments, the tubing 216 may fit over a protruding port, and in some embodiments, the tubing 216 may be connected to the male luer connector portion 210 with no friction coupling of the first end 231 being received within or being fit over the first port 218. When the first end 231 is coupled to the first port 218, the tubing is fluidly coupled to the lumen 212 through the first port 218.

In embodiments, the second end 233 is preferably configured to be coupled to a second port 236 of the female luer connector portion 220. The second end 233 may be sized to fit within the second port 236 and secured by adhesive, ultrasonic welding, or some other means of coupling. In some embodiments, the tubing 216 may fit over a protruding port, and in some embodiments, the tubing 216 may be connected to the female luer connector portion 220 with no friction coupling of the second end 233 being received within or being fit over the second port 236. When the second end 233 is coupled to the second port 236, the tubing is fluidly coupled to the lumen 228 through the second port 236. Accordingly, with the first end 231 being coupled to the first port 218, and the second end being coupled to the second port 236, fluid communication is provided between the male luer connector portion 210 lumen 212 and the female luer connector portion 220 lumen 228 through the first and second ports 218, 236, and through the tubing 216. In this configured, the lumens 212, 228, the first and second ports 218, 236, and/or the tubing 216 can be configured, through their characteristics (e.g., length, diameters, sizing, etc.) to control fluid flow between the lumens 212, 228 and reduce hemolysis through the total blood flow pathway from PIVC to blood collection device 40. As illustrated, the flow restriction device is configured to induce a resistance to the fluid flow. By inducing a resistance to the fluid flow, the rate of fluid flow through the assembly from PIVC to blood collection device is reduced. Reducing the flow rate of a fluid, such as blood, through the assembly can reduce the hemolysis index of the blood.

FIGS. 6A-6B illustrate embodiments of a flow restriction device 300 that have similar structures and functions to embodiments illustrated in FIGS. 1-5B. The flow restriction device includes a male luer connector portion 310, a middle connector portion 315, and a female luer connector portion 320. The middle connector portion 315 preferably includes an extended skirt or hollow chamber 319 that is configured to extend between the male luer connector portion 310 and the female luer connector portion 320. The hollow chamber 319 is preferably an extension of either the male luer connector portion 310 or the female luer connector portion 320 and is configured to couple with the other of either the male luer or female luer connector portions 310, 320.

The hollow chamber 319 can function in a similar manner as the core 222 of the embodiments of FIGS. 5A-5B in that the chamber 319 is configured to hold tubing 316 extending and conducting fluid between the male and female luer connector portions 310, 320. The hollow chamber 319 includes an internal surface 321 that is configured to contain the tubing 316 within the hollow chamber 319. As illustrated, the tubing 316 is preferably coiled as it extends through the chamber 319. The tubing 316 is preferably a flexible tubing that is capable of being coiled within the hollow chamber 319, but the tubing 316 can also be arranged within the hollow chamber 319 in other ways (e.g., straight, undulating, tortuous, etc.).

The tubing 316 preferably includes a first end 331 and a second end 333, and the first end 331 is configured to couple to a first port 318 of the male luer connector portion 310 as described in other embodiments herein. The second end 333 is configured to couple to a second port 336 of the female luer connector portion 320 as also describe in other embodiments herein. Accordingly, when the tubing 316 is coupled to the male and female luer connector portions 310, 320, fluid communication is provided from a lumen 312 of the male luer connector portion 310 to a lumen 328 of the female luer connector portion 320 through the first and second ports 318, 336 and the tubing 316.

Therefore, in some embodiments, the devices 100, 200, and 300 can direct fluid flow from the male luer connector portion into the fluid collection device 40 via the female luer connector portion. As depicted, the devices 100, 200, and 300 may provide fluid pathways from the catheter assembly 50 to the fluid collection device 40 that function to provide flow restriction. For example, in some embodiments, a leg 72 of the Y-adapter 70 may be coupled to the flow restriction devices 100, 200, and/or 300. The leg 72 of Y-adapter 70 may include a lumen into which the distal end of the male luer connector portion 110 may be coupled. The Y-adapter 70 may provide fluid-flow continuity between the flow restriction devices via a connector 90, which is depicted as a needleless connector, and the channel 135 with the catheter assembly 50, for example, via the extension tubing 60. Accordingly, the devices 100, 200, and/or 300 may define fluid pathways with a helical, spiral, extended, or otherwise indirect path (as discussed herein) through which fluid entering the flow restriction device from the catheter assembly 50, may flow through the flow restriction device for collection in the fluid collection device 40. For example, where blood is being withdrawn or collected from a patient, the medical fluid may be blood, and the fluid collection device 40 may be a blood collection device. In some embodiments, the blood collection device may be a blood collection tube holder such as the BD Vacutainer® LuerLok™ Access Device (LLAD). Accordingly, during blood collection or withdrawal from the patients, the blood sample may flow from the distal end of the male luer connector portion into the LLAD 40 via the flowpath.

The devices 100, 200, and/or 300 of the various embodiments described herein are advantageous over currently existing PIVC blood collection systems. For example, during blood draws with currently existing PIVCs, blood cells may experience supraphysiologic levels of shear stress as they flow from the distal end to the proximal end of the blood collection systems. Subjecting red blood cells to supraphysiologic levels of shear stress is considered a major source of mechanical damage and hemolysis of red blood cells. The diameter and effective length of the fluid pathways in the channel 135 or tubing 216, 316 may facilitate increased flow resistance within the vascular access system to reduce the flow rate and reduce shear stress experienced by the red blood cells 15. For example, the minimized diameter and elongated effective length of the fluid pathways may provide increased resistance to flow of the blood 15 and thereby decrease blood flow rate within the PIVC, device, and blood collection device assembly. Since the decreased blood flow rate causes a reduction in shear stress experienced by the red blood cells, the risk of hemolysis during blood collection may advantageously be reduced. Further, the geometry of the channel 135 or tubing 216, 316 can reduce the amount of blood wasted by currently existing PIVC blood collection systems.

Further, the flow restriction devices can reduce hemolysis without the use of active devices such as valves or other flow control devices. In some applications, the configuration of the flow restriction devices as described herein can minimize flushing of the vascular access device 10, minimize dead volume, and facilitate a compact design. Further, the configuration of the flow restriction devices can facilitate case of manufacturing compared to certain prior art designs. For example, in some applications, some embodiments provide that the male luer connector portion 110, 310 and the female luer connector portion 120, 320 can be joined together followed by ultrasonic welding to form the flow restriction devices 100, 300.

The fluid connector devices and associated blood collection systems of the various embodiments described herein additionally provide further advantages over currently existing blood collection systems. For example, add-on fluid connector devices described herein allow for hemolysis-reduction function to be integrated for PIVC blood draw. Further, the fluid connector devices described herein are compatible with PIVC placement and allow for seamless blood draw at insertion. Ion some embodiments, the fluid connector devices have the potential to stay inline throughout PIVC indwell for multiple blood draws. Additionally, since the fluid connector devices are an add-on which can be easily incorporated without any changes to existing PIVC, there is minimal impact to clinical setting and operations.

An optimized fluid pathway, also referred to herein as a first fluid pathway or flowpath or micro-channel, can be configured for providing a restricted flow rate for reducing hemolysis, and can have features including, but not limited to a tubular fluid pathway, a cannula, a lumen, a continuous non-linear channel, a groove, a fluid channel, and the like.

The fluid pathway can have a length that is selected based on one or more of the following: a gauge of a particular catheter, a particular catheter assembly configuration, or a clinical setup. In some embodiments, the optimized fluid pathway may include a length L from the female luer adapter 101 to the male luer adapter 102. In some embodiments, the optimized fluid pathway may include an inner diameter D.

Fluid flow in a tubular fluid pathway therethrough can be analyzed using Poiseuille's equation:

$$Q = \frac{π {D}^{4} Δ P}{128 μ L} = \frac{Δ P}{{R}_{f}}$$

where ΔP is a change in pressure gradient across the length of the fluid pathway, D and L are the inner diameter and length, respectively, of the fluid pathway, μ is the viscosity of a fluid, and

$${R}_{f} = \frac{128 μ L}{π {D}^{4}}$$

is the fluid resistance. Since μ is the viscosity of the fluid and not part of the extension tube geometry, a geometric factor G_{f} is defined such that R_{ƒ} (the fluid resistance) is

$${R}_{f} = \frac{128 μ}{π} {G}_{f} , where {G}_{f} = \frac{L}{{D}^{4}} .$$

In some embodiments, the optimized fluid pathway may have multiple sections with lengths (L1, L2, L3) and inner diameters of (D1, D2, D3), the geometric factor is then:

$${G}_{f} = {\int }_{0}^{L} \frac{dl}{{D ( l )}^{4}}$$

In some embodiments, the optimized fluid pathway may have a cross section that is not circular or complicated inside diameter profile. The geometric factor can be determined by measuring the flow rate (Q) at given pressure (ΔP) with known viscosity (μ) fluid:

$${G}_{f} = \frac{π Δ P}{128 μ Q}$$

The G_{f} value of the optimized fluid pathway may be selected to reduce the max shear stress for each catheter gauge to be the same or less than the max shear stress of a BD 21G VACUTAINER®UltraTouch™ push button blood collection set, which was previously considered the gold standard for blood draws. In some embodiments, Gr value of the optimized fluid pathway may be selected to reduce the max shear stress for each catheter gauge to be the same or less than the max shear stress of a BD 25G VACUTAINER®UltraTouch™ push button blood collection set.

In some embodiments, and by way of non-limiting example, an optimized fluid pathway can have a diameter of approximately 0.014 inch. In another non-limiting example, a cross-sectional area of an optimized fluid pathway is approximately 0.000152 inch².

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1. A flow restriction device, comprising: a male luer connector portion defining a first lumen; a female luer connector portion defining a second lumen; and a middle connector portion comprising a flexible tubing with a first end and a second end, the first end configured to fluidly couple with the first lumen and the second end configured to fluidly couple with the second lumen; wherein the tubing is helically arranged between the male luer connector portion and the female luer connector portion and comprises a fluid passage that is configured to provide fluid communication between the first lumen and the second lumen.

Clause 2. The flow restriction device of Clause 1, wherein the middle connector portion comprises a solid core extending between the male and female luer connector portions about which the tubing is supported.

Clause 3. The flow restriction device of Clause 2, wherein the tubing is helically wound around the core of the middle connector portion.

Clause 4. The flow restriction device of Clause 1, wherein the fluid passage comprises a length and a diameter that is configured to increase flow resistance through the tubing to reduce shear stress on the fluid.

Clause 5. The flow restriction device of Clause 1, wherein the middle connector portion comprises a hollow chamber within which the tubing extends between the male and female luer connector portions.

Clause 6. The flow restriction device of Clause 5, wherein the tubing is helically wound within the hollow chamber.

Clause 7. The flow restriction device of Clause 1, wherein the first end of the tubing is coupled to the first lumen through a first port formed in the male luer connector portion, and the second end of the tubing is coupled to the second lumen through a second port formed in the female luer connector portion.

Clause 8. A flow restriction device, comprising: a first connector portion defining a first lumen; a second connector portion defining a second lumen; and a third connector portion comprising a tubing with a first end and a second end, the first end configured to fluidly couple with the first lumen and the second end configured to fluidly couple with the second lumen, wherein the tubing comprises a fluid passage that is configured to provide fluid communication between the first lumen and the second lumen.

Clause 9. The flow restriction device of Clause 8, wherein the third connector portion is configured to extend between the first and second connector portions and comprises a core about which the tubing is supported.

Clause 10. The flow restriction device of Clause 9, wherein the tubing is flexible and is helically wound around the core of the third connector portion.

Clause 11. The flow restriction device of Clause 9, wherein the core is a solid core that does not conduct fluid and comprises a helically wound groove that provides a trough and ridges to secure placement of the tubing.

Clause 12. The flow restriction device of Clause 8, wherein the fluid passage comprises a length and a diameter that is configured to increase flow resistance through the tubing sufficient to reduce shear stress on the fluid.

Clause 13. The flow restriction device of Clause 8, wherein the third connector portion comprises a hollow chamber within which the tubing extends between the first and second connector portions.

Clause 14. The flow restriction device of Clause 13, wherein the tubing is helically wound within the hollow chamber.

Clause 15. The flow restriction device of Clause 8, wherein the first end of the tubing is coupled to the first lumen through a first port formed in the first connector portion, and the second end of the tubing is coupled to the second lumen through a second port formed in the second connector portion.

Clause 16. The flow restriction device of Clause 8, wherein at least one of the first connector portion and second connector portion comprises a luer connector.

Clause 17. The flow restriction device of Clause 8, wherein the third connector portion extends between the first and second connector portions.

Clause 18. A peripheral intravenous catheter assembly configured to limit hemolysis during drawing of blood from a patient, comprising: a catheter hub having a proximal end and a distal end; a fluid collection device; and a flow restriction device, comprising: a male luer connector portion defining a first lumen; a female luer connector portion defining a second lumen; and a middle connector portion comprising a flexible tubing with a first end and a second end, the first end configured to fluidly couple with the first lumen and the second end configured to fluidly couple with the second lumen; wherein the tubing is arranged between the male luer connector portion and the female luer connector portion and comprises a fluid passage that (i) is configured to provide fluid communication between the first lumen and the second lumen and (ii) comprises a length and a diameter that is configured to increase flow resistance through the tubing to reduce shear stress on the fluid.

Clause 19. The peripheral intravenous catheter assembly of Clause 18, wherein the middle connector portion comprises a solid core extending between the male and female luer connector portions about which the tubing is supported and is helically wound.

Clause 20. The peripheral intravenous catheter assembly of Clause 18, wherein the middle connector portion comprises a hollow chamber within which the tubing is helically wound between the male and female luer connector portions.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, or operations in the processes or methods disclosed are illustrations of exemplary approaches. Based upon implementation preferences or scenarios, it is understood that the specific order or hierarchy of steps, operations or processes may be rearranged. Some of the steps, operations or processes may be performed simultaneously. In some implementation preferences or scenarios, certain operations may or may not be performed. Some or all of the steps, operations, or processes may be performed automatically, without the intervention of a user. The accompanying method claims present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but are to be accorded the full scope consistent with the language of the claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A flow restriction device, comprising:
a male luer connector portion defining a first lumen;
a female luer connector portion defining a second lumen; and
a middle connector portion comprising a flexible tubing with a first end and a second end, the first end configured to fluidly couple with the first lumen and the second end configured to fluidly couple with the second lumen;
wherein the tubing is helically arranged between the male luer connector portion and the female luer connector portion and comprises a fluid passage that is configured to provide fluid communication between the first lumen and the second lumen.

2. The flow restriction device of claim 1, wherein the middle connector portion comprises a solid core extending between the male and female luer connector portions about which the tubing is supported.

3. The flow restriction device of claim 2, wherein the tubing is helically wound around the core of the middle connector portion.

4. The flow restriction device of claim 1, wherein the fluid passage comprises a length and a diameter that is configured to increase flow resistance through the tubing to reduce shear stress on the fluid.

5. The flow restriction device of claim 1, wherein the middle connector portion comprises a hollow chamber within which the tubing extends between the male and female luer connector portions.

6. The flow restriction device of claim 5, wherein the tubing is helically wound within the hollow chamber.

7. The flow restriction device of claim 1, wherein the first end of the tubing is coupled to the first lumen through a first port formed in the male luer connector portion, and the second end of the tubing is coupled to the second lumen through a second port formed in the female luer connector portion.

8. A flow restriction device, comprising:
a first connector portion defining a first lumen;
a second connector portion defining a second lumen; and
a third connector portion comprising a tubing with a first end and a second end, the first end configured to fluidly couple with the first lumen and the second end configured to fluidly couple with the second lumen,
wherein the tubing comprises a fluid passage that is configured to provide fluid communication between the first lumen and the second lumen.

9. The flow restriction device of claim 8, wherein the third connector portion is configured to extend between the first and second connector portions and comprises a core about which the tubing is supported.

10. The flow restriction device of claim 9, wherein the tubing is flexible and is helically wound around the core of the third connector portion.

11. The flow restriction device of claim 9, wherein the core is a solid core that does not conduct fluid and comprises a helically wound groove that provides a trough and ridges to secure placement of the tubing.

12. The flow restriction device of claim 8, wherein the fluid passage comprises a length and a diameter that is configured to increase flow resistance through the tubing sufficient to reduce shear stress on the fluid.

13. The flow restriction device of claim 8, wherein the third connector portion comprises a hollow chamber within which the tubing extends between the first and second connector portions.

14. The flow restriction device of claim 13, wherein the tubing is helically wound within the hollow chamber.

15. The flow restriction device of claim 8, wherein the first end of the tubing is coupled to the first lumen through a first port formed in the first connector portion, and the second end of the tubing is coupled to the second lumen through a second port formed in the second connector portion.

16. The flow restriction device of claim 8, wherein at least one of the first connector portion and second connector portion comprises a luer connector.

17. The flow restriction device of claim 8, wherein the third connector portion extends between the first and second connector portions.

18. A peripheral intravenous catheter assembly configured to limit hemolysis during drawing of blood from a patient, comprising:
a catheter hub having a proximal end and a distal end;
a fluid collection device; and
a flow restriction device, comprising:
a male luer connector portion defining a first lumen;
a female luer connector portion defining a second lumen; and
a middle connector portion comprising a flexible tubing with a first end and a second end, the first end configured to fluidly couple with the first lumen and the second end configured to fluidly couple with the second lumen;
wherein the tubing is arranged between the male luer connector portion and the female luer connector portion and comprises a fluid passage that (i) is configured to provide fluid communication between the first lumen and the second lumen and (ii) comprises a length and a diameter that is configured to increase flow resistance through the tubing to reduce shear stress on the fluid.

19. The peripheral intravenous catheter assembly of claim 18, wherein the middle connector portion comprises a solid core extending between the male and female luer connector portions about which the tubing is supported and is helically wound.

20. The peripheral intravenous catheter assembly of claim 18, wherein the middle connector portion comprises a hollow chamber within which the tubing is helically wound between the male and female luer connector portions.
